(19) United States
(12) Patent Application Publication
(10) Pub. No.: US 2011/0052548 A1
(43) Pub. Date: Mar. 3, 2011

(54) CELL CULTURE MEDIA TO DIFFERENTIATE EMBRYONIC STEM CELLS INTO NEURONAL LINEAGES

(75) Inventors: Wei-Chun Chin, Merced, CA (US); Eric Yi-Tong Chen, Merced, CA (US); Tzu-I Chao, Merced, CA (US); Chi-Shuo Chen, Merced, CA (US)

(73) Assignee: The Regents of the University of California

(21) Appl. No.: 12/869,599

(22) Filed: Aug. 26, 2010

Related U.S. Application Data
(60) Provisional application No. 61/237,649, filed on Aug. 27, 2009.

(51) Int. Cl. A61K 35/12; C12Q 1/02; C12N 5/00; C12N 5/0735; C12N 5/071; C12N 5/02; A61P 25/00

(52) U.S. Cl. 424/93.7; 435/29; 435/325; 435/354; 435/363; 435/366; 435/377; 435/404

(57) ABSTRACT

This invention provides compositions for differentiating an isolated embryonic stem cell or an isolated embryoid body into neuronal progenitor cells and methods for using same.

### CROSS-REFERENCE TO RELATED APPLICATION

This application claims the benefit under 35 U.S.C. §119(e) of U.S. Provisional Application No. 61/237,649, filed Aug. 27, 2009, the contents of which are hereby incorporated by reference into the present disclosure.

### BACKGROUND

Throughout this disclosure, various technical and patent publications are referenced to more fully describe the state of the art to which this invention pertains. These publications are incorporated by reference, in their entirety, into this application.

Stem cells are capable of self-renewal through numerous cycles of cell divisions and capable of transformation into specialized cell types. The ultimate cell type into which the stem cells will differentiate depends on intrinsic regulatory factors and the microenvironment. Lee et al. (2005) Tissue Eng. and Reg. Med. 2(3):264-273.

Stem cells are typically classified into several types: embryonic stem cells (ESCs) found in blastocysts, adult stem cells found in post-embryonic tissues and induced pluripotent stem cells (iPSCs) which have been de-differentiated from the adult type into an embryonic-type state. ESCs are self-renewal and pluripotent cells derived from the inner cell mass (ICM) of blastocysts within 14 days after fertilization. Adult stem cells are extracted from fetal and adult tissue and, for the most part, are limited in the cell types into which they can differentiate. Although they have limited differentiating capacity as compared to ESC, the adult stem cells can function stably and have been shown to differentiate across some tissue types. Pittenger et al. (1999) Science 284(2):143-147 and Huard et al. (2004) Curr. Opin. Biotechnol. 15(5):419-23.

Adult stem cells that can be utilized to treat nervous-system diseases are neural stem cells. However, because these stem cells exist in specific regions of the brain, such as the subventricular zone (SVZ) and the hippocampus, it is impossible to isolate them in therapeutically sufficient amounts. Bone marrow-derived mesenchymal stem cells, muscle-derived stem cells and adipose-derived stem cells are advantageous in that they exhibit in vitro self-renewing abilities and can be easily isolated and cultured as adult stem cells capable of differentiating into bones, cartilages and adipose tissues under adequate conditions for differentiation.

Embryonic stem cells also have been differentiated into neural cells but the techniques have been hampered by the inability to obtain homogenous populations and the glial cell subtype (Suter et al. (2008) J. of Path. 215(4):355-368) and the availability of culture media.

Even with these limitations, neural stem and progenitor cells offer great potential for treatment of neurological disorders, e.g., Traumatic Brain Injury, Alzheimer's disease, Parkinson's disease, epilepsy, Huntington's disease, and stroke. Cell populations that can reconstitute the neural network is key to realizing these therapies.

Methods and compositions for differentiating adult and embryonic stem cell populations to neural cell populations are known in the art and described in U.S. Patent Publ. No. 2009/0035284 which itself teaches the differentiation of embryonic stem cells into a homogeneous population of neural stem cells with unlimited self-renewal capability. U.S. Pat. No. 7,011,828 and U.S. Patent Publ. Nos. 2005/0260747 A1 and 2006/0078543 A1 are reported to teach the proliferation of an enriched population of embryonic stem cells, which are induced to differentiate in vitro to neural progenitor cells, neurons, and/or glial cells. U.S. Pat. No. 6,887,706 teaches a method of differentiating embryonic stem cells into neural precursor cells using the growth factor FGF2. In vitro differentiation of the ES cell-derived neural precursors was induced by withdrawal of FGF2 and plating on ornithine and laminin substrate.

U.S. Pat. No. 7,015,037 and U.S. Patent Publication No. 2006/0030041 A1 are reported to teach the differentiation of multipotent adult stem cells (MASCs) to form glial, neuronal, or oligodendrocyte cell types using growth factors, chemokines, and cytokines such as EGF, PDGF-BB, FGF2, and FGF-9. U.S. Patent Publication No. 2006/0252149 is alleged to disclose the maintenance of central nervous system (CNS) cells in vitro that retain the ability to proliferate and remain in an undifferentiated state by culturing in the presence of soluble laminin alone or together with one or more laminin associated factors (LAFs) and one or more of the CNS mitogens EGF, bFGF (also referred to as FGF2), and LIF.

U.S. Patent Publ. No. 2007/0059823 A1 discloses a method for inducing ES cells and MASCs to differentiate into neuronal cells by culturing the stem cells initially with bFGF and later with FGF8, Sonic Hedgehog, brain-derived neutrotrophic factor, and astrocytes. Supplementing the media with growth factors such as EGF, platelet derived growth factor (PDGF), and LIF keeps the cells in an undifferentiated state.

U.S. Patent Publ. No. 2005/0214941 A1 teaches a method for improving the growth rate of human fetal brain stem cells by culturing human neural stem cells (hNSCs) with bFGF, EGF, and LIF. Culturing the hNSCs on a surface coated with polyornithine and fibronectin increases the rate of proliferation of neural stem cell cultures and increasing the number of neurons.

The technical literature also reports methods and compositions to culture neural stem cells. For example, Ling et al. (1998) Exp. Neurol. 149:411, reports the isolation of progenitor cells from the germinal region of rat fetal mesencephalon. The cells were grown in EGF, and plated on poly-lysine coated plates, whereupon they formed neurons and glia, with occasional tyrosine hydroxylase positive (dopaminergic) cells, enhanced by including IL-1, IL-11, LIF, and GDNF in the culture medium.

Wagner et al. (1999) Nature Biotechnol. 17:653, reports the induction of cells with a ventral mesencephalic dopaminergic phenotype from an immortalized multipotent neural stem cell line. The cells were transfected with a Nurr1 expression vector, and then cocultured with VM type 1 astrocytes. Over 80% of the cells obtained were claimed to have a phenotype resembling endogenous dopaminergic neurons.

Ding et al. (2006) Angewandte Chemie. 118(4):605-607 reports the differentiation of neural progenitor cells into nerve cells using a small molecule neuropathiazol. These cells were further reported to restore damaged tissues and facilitate the growth of intrinsic nerve cells in an animal model of nervous-system diseases. Shetty et al. (2007) Stem Cells 25(8):2014-2017.

However, none of the foregoing patents, patent publications and/or technical literature teach a simple and economical method to manufacture culture media that differentiate embryonic stem cells and/or embryoid bodies into neuronal lineages. Traditionally, differentiation is a time consuming, labor intensive and very expensive process where a cocktail of growth factors have been heavily used to differentiate embryonic stem cells and/or embryoid bodies into neuronal lineages. The differentiation process takes more than 2 to 3 weeks. Therefore, a more direct, simple and inexpensive approach is needed to expedite and reduce the cost of differentiating embryonic stem cells and/or embryoid bodies into neuronal cell lineages. This invention addresses and satisfies this need and provides related advantages as well.

### SUMMARY OF THE INVENTION

Embryonic stem cells and embryoid bodies differentiating into neuronal lineages is vastly used in academic research and commercial sectors. A quick, stable and vast manufacturing of neuronal lineages are critical to repair neurological disorders, spinal cord injuries and neuronal diseases. This invention provides a simple and easy way to meet the demands of quick and high yield production of neuronal lineage cell for both research institutions and industries.

Thus, in one aspect, this invention provides a neuronal growth media supplement comprising, or alternatively consisting essentially of, or yet further consisting of an independent effective amount of from about 10 nM to about 100 μM of each of ferric chloride, ferrous chloride, ferri citrate, citric acid ammonium ferric citrate and ATP. In another aspect, this invention provides a composition for differentiating an isolated embryonic stem cell, an iPSC, a parthenogenetic stem cell or an isolated embryoid body into neuronal progenitor cells or neurons comprising, or alternatively consisting essentially of, or yet further consisting of, an effective amount of a cell culture or growth medium and from about 10 nM to about 100 μM of each of ferric chloride, ferrous chloride, ferri citrate, citric acid ammonium ferric citrate and ATP. Methods for preparing the compositions are further provided herein. In one aspect, the composition differentiates the stem cells into a substantially homogenous population of the neuronal progenitor cells or neurons.

This invention also provides an in vitro method for differentiating an isolated embryonic stem cell, an iPSC, a parthenogenetic stem cell, or an isolated embryoid body into neuronal progenitor cells or neurons comprising contacting an effective amount of a composition of this invention or from about 10 nM to about 100 μM of each of ferric chloride, ferrous chloride, ferri citrate, citric acid ammonium ferric citrate and ATP with the isolated embryonic stem cell, the iPSC, a parthenogenetic or an isolated embryoid body, under suitable conditions and for an effective amount of time. In one aspect, the composition differentiates the stem cells into a substantially homogenous population of the neuronal progenitor cells or neurons.

Further provided is a kit for use in preparing a population of neuronal progenitor cells comprising, or alternatively consisting of, or yet further consisting of, an effective amount of the composition or supplement of this invention and instructions for use of the composition.

### DETAILED DESCRIPTION OF THE INVENTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis (1989) Molecular Cloning: A Laboratory Manual, 2^{nd} edition; F. M. Ausubel, et al. eds. (1987) Current Protocols In Molecular Biology; the series Methods in Enzymology (Academic Press, Inc.): PCR 2: A Practical Approach (1995) (M. J. MacPherson, B. D. Hames and G. R. Taylor eds.); Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source and which allow the manipulation of the material to achieve results not achievable where present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells or tissues that are isolated from other cells or tissues and is meant to encompass both cultured and engineered cells or tissues.

As used herein, “stem cell” defines a cell with the ability to divide for indefinite periods in culture and give rise to specialized cells. At this time and for convenience, stem cells are categorized as somatic (adult), embryonic or induced pluripotent stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. Non-limiting examples of embryonic stem cells are the HES2 (also known as ES02) cell line available from ESI, Singapore and the H1 or H9 (also know as WA01) cell line available from WiCell, Madison, Wis. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4. An -induced pluripotent stem cell (iPSC) is an artificially derived stem cell from a non-pluripotent cell, typically an adult somatic cell, produced by inducing expression of one or more stem cell specific genes.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6 (2003).

“Embryoid bodies or EBs” are three-dimensional (3-D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of neuronal progenitor cells or neuronal cells.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

As used herein, the term “differentiates or differentiated” defines a cell that takes on a more committed (“differentiated”) position within the lineage of a cell. “Dedifferentiated” defines a cell that reverts to a less committed position within the lineage of a cell.

As used herein, a “pluripotent cell” defines a less differentiated cell that can give rise to at least two distinct (genotypically and/or phenotypically) further differentiated progeny cells.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include biocompatible scaffolds, pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

A “biocompatible scaffold” refers to a scaffold or matrix for tissue-engineering purposes with the ability to perform as a substrate that will support the appropriate cellular activity to generate the desired tissue, including the facilitation of molecular and mechanical signaling systems, without eliciting any undesirable effect in those cells or inducing any undesirable local or systemic responses in the eventual host. In other embodiments, a biocompatible scaffold is a precursor to an implantable device which has the ability to perform its intended function, with the desired degree of incorporation in the host, without eliciting an undesirable local or systemic effects in the host. Biocompatible scaffolds are described in U.S. Pat. No. 6,638,369.

A neuron is an excitable cell in the nervous system that processes and transmits information by electrochemical signaling. Neurons are found in the brain, the vertebrate spinal cord, the invertebrate ventral nerve cord and the peripheral nerves. Neurons can be identified by a number of markers that are listed on-line through the National Institute of Health at the following website: “stemcells.nih.gov/info/scireport/appendixe.asp#eii,” and are commercially available through Chemicon (now a part of Millipore, Temecula, Calif.) or Invitrogen (Carlsbad, Calif.). For example, neurons may be identified by expression of neuronal markers B-tubulin III (neuron marker, Millipore, Chemicon), Tuj 1 (beta-III-tubulin); MAP-2 (microtubule associated protein 2, other MAP genes such as MAP-1 or -5 may also be used); anti-axonal growth clones; ChAT (choline acetyltransferase (motoneuron marker, Millipore, Chemicon); Olig2 (motorneuron marker, Millipore, Chemicon), Olig2 (Millipore, Chemicon), CgA (anti-chromagranin A); DARRP (dopamine and cAMP-regulated phosphoprotein); DAT (dopamine transporter); GAD (glutamic acid decarboxylase); GAP (growth associated protein); anti-HuC protein; anti-HuD protein; alpha-internexin; NeuN (neuron-specific nuclear protein); NF (neurofilament); NGF (nerve growth factor); gamma-NSE (neuron specific enolase); peripherin; PH8; PGP (protein gene product); SERT (serotonin transporter); synapsin; Tau (neurofibrillary tangle protein);anti-Thy-1; TRK (tyrosine kinase receptor); TRH (tryptophan hydroxylase); anti-TUC protein; TH (tyrosine hydroxylase); VRL (vanilloid receptor like protein); VGAT (vesicular GABA transporter), VGLUT (vesicular glutamate transporter).

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller (2006) Brain Res. 1091(1):258-264; Pluchino et al. (2005) Brain Res. Brain Res. Rev. 48(2):211-219; and Goh et al. (2003) Stem Cell Res. 12(6):671-679. Neural stem cells can be identified and isolated by neural stem cell specific markers including, but limited to, CD133, ICAM-1, MCAM, CXCR4 and Notch 1. Neural stem cells can be isolated from animal or human by neural stem cell specific markers with methods known in the art. See, e.g., Yoshida et al. (2006) Stem Cells 24(12):2714-22.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell include, without limitation, a progenitor nerve cell.

A “neural precursor cell”, “neural progenitor cell” or “NP cell” refers to a cell that has a capacity to differentiate into a neural cell or neuron. A NP cell can be an isolated NP cell, or derived from a stem cell including but not limited to an iPS cell. Neural precursor cells can be identified and isolated by neural precursor cell specific markers including, but limited to, nestin and CD133. Neural precursor cells can be isolated from animal or human tissues such as adipose tissue (see, e.g., Vindigni et al. (2009) Neurol. Res. 2009 Aug. 5. [Epub ahead of print]) and adult skin (see, e.g., Joannides (2004) Lancet. 364(9429):172-8). Neural precursor cells can also be derived from stem cells or cell lines or neural stem cells or cell lines. See generally, e.g., U.S. Patent Application Publications Nos.: 2009/0263901, 2009/0263360 and 2009/0258421.

A nerve cell that is “terminally differentiated” refers to a nerve cell that does not undergo further differentiation in its native state without treatment or external manipulation. In one embodiment, a terminally differentiated cell is a cell that has lost the ability to further differentiate into a specialized cell type or phenotype.

A population of cells intends a collection of more than one cell that is identical (clonal) or non-identical in phenotype and/or genotype.

The term neurodegenerative condition (or disorder) is an inclusive term encompassing acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

Other neurodegenerative conditions include dementias, regardless of underlying etiology, including age-related dementia and other dementias and conditions with memory loss including dementia associated with Alzheimer's disease, vascular dementia, diffuse white matter disease (Binswanger's disease), dementia of endocrine or metabolic origin, dementia of head trauma and diffuse brain damage, dementia pugilistica and frontal lobe dementia.

The term treating (or treatment of) a neurodegenerative disorder or condition refers to ameliorating the effects of, or delaying, halting or reversing the progress of, or delaying or preventing the onset of, a neurodegenerative condition as defined herein.

The term effective amount refers to a concentration or amount of a reagent or composition, such as a composition as described herein, cell population or other agent, that is effective for producing an intended result, including cell growth and/or differentiation in vitro or in vivo, or for the treatment of a neurodegenerative condition as described herein. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or composition to achieve its intended result, e.g., the differentiation of cells to a pre-determined cell type.

The term patient or subject refers to animals, including mammals, preferably humans, who are treated with the pharmaceutical compositions or in accordance with the methods described herein.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Additionally, when the purpose of the experiment is to determine if an agent effects the differentiation of a stem cell, it is preferable to use a positive control (a sample with an aspect that is known to affect differentiation) and a negative control (an agent known to not have an affect or a sample with no agent added).

The term patient or subject refers to animals, including mammals, preferably humans, who are treated with the pharmaceutical compositions or in accordance with the methods described herein.

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

### Exemplary Embodiments

Applicants have discovered that the use of a composition comprising, or alternatively consisting essentially of, or yet further consisting of, an independent effective amount of a stem cell growth medium and from about 10 nM to about 100 μM of each of ferric chloride, ferrous chloride, ferri citrate, citric acid ammonium ferric citrate and ATP supports the differentiation of an isolated embryonic stem cell or an isolated embryoid body into neuronal progenitor cells. In one aspect, the amount of each of the ferric chloride, ferrous chloride, ferri citrate, citric acid ammonium ferric citrate and ATP may be the same or different from each other and is from about 20 nM to about 50 μM, or alternatively about 30 nm to about 40 μM, or alternatively about 50 nm to about 20 μM, or alternatively about 10 nm to about 100 μM, or alternatively about 100 nm to about 100 μM, or alternatively about 250 nm to about 100 μM, or alternatively about 500 nm to about 10 μM, or alternatively about 750 nm to about 40 μM, or alternatively about 30 nm to about 40 μM, or alternatively about 500 nm to about 10 μM, or alternatively about 250 nm to about 10 μM.

The components of ferric chloride, ferrous chloride, ferri citrate, citric acid ammonium ferric citrate are commercially available from, e.g., Sigma and ATP is commercially available from, e.g., Fluka.

In another aspect, this invention provides a neuronal growth media supplement comprising, or alternatively consisting essentially of, or yet further consisting of an independent effective amount of from about 10 nM to about 100 μM of each of ferric chloride, ferrous chloride, ferri citrate, citric acid ammonium ferric citrate and ATP. In one aspect, the amount of each of the ferric chloride, ferrous chloride, ferri citrate, citric acid ammonium ferric citrate and ATP may be the same or different from each other and is from about 20 nM to about 50 μM, or alternatively about 30 nm to about 40 μM, or alternatively about 50 nm to about 20 μM, or alternatively about 10 nm to about 100 μM, or alternatively about 100 nm to about 100 μM, or alternatively about 250 nm to about 100 μM, or alternatively about 500 nm to about 10 μM, or alternatively about 750 nm to about 40 μM, or alternatively about 30 nm to about 40 μM, or alternatively about 500 nm to about 10 μM, or alternatively about 250 nm to about 10 μM. In one aspect, the amount of each of the aforementioned components is equivalent and is from about 10 nm to about 100 μM or alternatively from about 100 nm to about 100 μM. In one aspect, the supplement components are pre-mixed to the effective amount and then added to the cells or culture media or alternatively the components are added to the cells in culture media in amount which brings the concentration to the effective amount.

A cell growth medium as used here intends any of the commercially available mediums or mediums known to those skilled in the art for the differentiation of embryonic or embryoid bodies. Examples of such are described in the literature in Chao et al. (2009) Biochem. Biophys. Res. Comm. 384:426-430; Dhara et al. (2008) J. Cell. Biochem. 105:633-640; and Gerrard et al. (2005) Stem Cells 23:1234-1241 or commercially available such as N2 supplement-bFGF which is commercially available from Invitrogen and Sigma, respectively. In one aspect, the cell growth media further comprises, or alternatively consists essentially of, or yet further consists of non-essential amino acids, e.g. MEM non-essential amino acids commercially available from Invitrogen (amino acids glycine, L-alanine, L-asparagine, L-aspartic acid, L-glutamic acid, L-proline and L-serine, 10 mM (100×) liquid solution). This is added to the medium in a dilution factor of about 1:50, or alternatively about 1:60; or alternatively about 1:70, or alternatively about 1:80, or alternatively about 1:90, or alternatively about 100, or alternatively about 1:110, or alternatively about 1:120. In one particular aspect the non-essential amino acids are added at a dilution of about 1:100.

The compositions of this invention are useful to differentiate an isolated embryonic stem cell, an embroyid bodies or an iPS stem cell to a neuronal progenitor or neuron or a population of neuronal progenitors or neurons. In one aspect, the population of cells provided by use of the compositions and methods of this invention are substantially homogeneous. The isolated stem cell is of animal origin, e.g., a mammalian cell, e.g., a human cell, a simian cell, a bovine cell or a murine cell. In one aspect, the isolated embryonic stem cell, iPSC or embryoid body is of human origin. The cells can be cultured cells, e.g., available from the American Tissue Culture Collection (ATCC, Bethesda Md. or Wicell, Madison. Wis., for example) or isolated from an animal or human subject using methods known to those skilled in the art.

In a further aspect, the composition or supplement comprises, or alternatively consists essentially of, or yet further consists of, an additional carrier, e.g., a pharmaceutically acceptable carrier.

This invention also provides a method of preparing the composition by admixing an independently effective amount of a stem cell growth medium and from about 10 nM to about 100 μM of each of ferric chloride, ferrous chloride, ferri citrate, citric acid ammonium ferric citrate and ATP. In one aspect, the amount of each of the ferric chloride, ferrous chloride, ferri citrate, citric acid ammonium ferric citrate and ATP is may be the same or different from each other and can be in an amount from about 20 nM to about 50 μM, or alternatively about 30 nm to about 40 μM, or alternatively about 50 nm to about 20 μM, or alternatively about 10 nm to about 100 μM, or alternatively about 100 nm to about 100 μM, or alternatively about 250 nm to about 100 μM, or alternatively about 500 nm to about 10 μM, or alternatively about 250 nm to about 10 μM. In one aspect, the amount of each of the aforementioned components is equivalent and is from about 10 nm to about 100 μM or alternatively from about 100 nm to about 100 μM. In a further aspect, a carrier such as a pharmaceutically acceptable carrier can be added to the composition.

The compositions of the invention are useful to for differentiating, in vitro, an isolated embryonic stem cell, IPSC, a parthenogenetic stem cell, or an isolated embryoid body into neuronal progenitor cells or neurons or a population of same, by contacting an effective amount of a composition or the growth supplement as described above. In one aspect, the supplement components are pre-mixed to the effective amount and then added to the cells or culture media or alternatively the components are added to the cells in culture media in amount which brings the concentration to the effective amount. These compositions or components are contacted with the isolated embryonic stem cell, the iPSC, the parthenogenetic stem cell, or the isolated embryoid body and then cultured under suitable conditions and for an effective amount of time to differentiate the cells. For the purpose of illustration only, the effective amount of time includes, for example, for more than one day, or alternatively for two days, or for three days or more, prior to media change. Methods of determining when the cells have been differentiated are known in the art and include for example, the expression of neuronal-specific cell markers or physiological characteristics. The cell populations prepared by the methods and compositions of this invention are substantially homogeneous.

Also provided herein are the neuronal progenitor cells, neurons and cell populations of same obtained by use of the inventors' compositions and methods. The cells can be modified for use by incorporation of exogenous polynucleotides and/or polypeptides for further use. They can be combined with carriers such as pharmaceutically acceptable carrier or a biocompatible scaffold for use. The cells are useful in diagnostic assays to test drugs prior to administration to a patient or in therapeutic methods to treat neurodegenerative diseases or conditions by administering to a subject in need of such treatment an effective amount of the cells. For the purpose of illustration only, the subject is an animal, such as a mammal, e.g., a human, a murine, or a simian. In one aspect, the cells are autologous. In another aspect, the cells are allogeneic.

Also provided is a method for assaying a potential agent for the ability to affect cell migration, growth and/or differentiation of an isolated stem cell, comprising, or alternatively consisting essentially of, or yet further consisting of contacting the cell with an agent and the composition as described herein and allowing the cell to migrate or divide and then assaying for the agent's effect on the cell's migration, growth and/or differentiation. Suitable positive and negative controls can be cultured for the purpose of comparison. Assaying can be accomplished by any method known to those of skill in the art, e.g., visual observation with the naked eye or under a microscope or by use of differentiation specific markers.

For the purposes of this invention, an “agent” is intended to include, but not be limited to a biological or chemical compound such as a simple or complex organic or inorganic molecule, a peptide, a protein or an oligonucleotide. A vast array of compounds can be synthesized, for example oligomers, such as oligopeptides and oligonucleotides and synthetic organic compounds based on various core structures; these compounds are also included in the term “agent”. In addition, various natural sources can provide compounds for screening, such as plant or animal extracts and the like. It should be understood, although not always explicitly stated that the agent is used alone or in combination with another agent, having the same or different biological activity as the agents identified by the inventive screen. The agents and methods also are intended to be combined with other therapies.

When the agent is a nucleic acid, it can be added to the cell cultures by methods known in the art, which includes, but is not limited to calcium phosphate precipitation, microinjection or electroporation. Alternatively or additionally, the nucleic acid can be incorporated into an expression or insertion vector for incorporation into the cells. Vectors that contain both a promoter and a cloning site into which a polynucleotide can be operatively linked are well known in the art and briefly described in the appendix.

When the agent is a composition other than a DNA or RNA nucleic acid molecule, the suitable conditions comprise directly added to the cell culture or added to culture medium for addition. As is apparent to those skilled in the art, an “effective” amount must be added which can be empirically determined and must be administered for an effective amount of time.

This invention also provides the isolated population of cells, e.g., neurons prepared by the method of this invention as well as a composition comprising, or alternatively consisting essentially of, or yet further consisting of, the population of cells described above and a carrier, e.g., a pharmaceutically acceptable carrier. The population of cells may be substantially homogenous or substantially heterogeneous, depending on the cell of origin and culture conditions. In one aspect, the population of cells are differentiated from an embryoid body. A population of cells intends a plurality of cells of at least about 1×10⁶ cells, or alternatively at least 5×10⁵ cells, or alternatively at least 1×10⁵ cells.

The cells are useful in diagnostic and therapeutic applications as known to those of skill in the art. Accordingly, this invention also provides a method for treating a disease or disorder requiring substantially parallel cells, such as a neurological condition, by administering to a subject in need of the treatment an effective amount of the cells of this invention. Such disorders, include but are not limited to neurodegenerative disorders as defined herein, such as Traumatic Brain Injury, Alzheimer's disease, Parkinson's disease, epilepsy, Huntington's disease, and stroke. Cell populations that can reconstitute the neural network is key to realizing these therapies. The methods are accomplished by administering an effective amount of the cells. Modes of administration and the amount of cells necessary for the specific treatment. The cells may be autologous or allergenic to the subject receiving treatment.

In certain embodiments, the cells are administered with at least one other cell type, such as an astrocyte, oligodendrocyte, neuron, neural progenitor or other cell. Alternatively or in addition, the cells are administered with at least one other agent, such as a drug for neural therapy, or another beneficial adjunctive agent such as an anti-inflammatory agent, anti-apoptotic agents, antioxidant or growth factor. In these embodiments, the other agent can be administered simultaneously with, or before, or after, the neurons.

In certain embodiments, the cells are administered at a pre-determined site in the central or peripheral nervous system of the patient. They can be administered by injection or infusion, or encapsulated within an implantable device, or by implantation of a matrix or scaffold containing the cells.

In certain embodiments, the pharmaceutical composition is formulated for administration by injection or infusion. Alternatively, it may comprise an implantable device in which the cells are encapsulated, or a matrix or scaffold containing the cells.

According to yet another aspect of the invention, a kit is provided for treating a patient having a neurodegenerative condition. The kit comprises a pharmaceutically acceptable carrier, a population of the above-described cells and instructions for using the kit in a method of treating the patient. The kit may further comprises at least one reagent and instructions for culturing the cells. It can also comprise, or alternatively consisting of, or yet further consisting of, a population of at least one other cell type, or at least one other agent for treating a neurodegenerative condition.

This invention also provides a kit for use in preparing a population of neuronal progenitor cells comprising an effective amount of the composition and/or supplement as described herein and instructions for use of the composition or supplement.

### Example

The present technology is further understood by reference to the following example. The present technology is not limited in scope by the examples, which are intended as illustrations of aspects of the present technology. Any methods that are functionally equivalent are within the scope of the present technology. Various modifications of the present technology in addition to those described herein will become apparent to those skilled in the art from the foregoing description. Such modifications fall within the scope of the appended claims.

To make the composition, the ferric chloride, ferrous chloride, ferric citrate, citric acid, ammonium ferric citrate and ATP were purchased in salt/powder form and subsequently reconstituted in double de-ionized water to make stock solutions. 100 mM of each solutions were prepared for each and can be added to the growth media or alternatively directly diluted in each well containing stem cell with medium. The supplemented media were replaced and replenished with fresh supplement media every two days.

Human Embryonic Stem Cell (hESC) lines H9 obtained from Wicell (Madison, Wis.) (passage 32-55) were cultured in a 20% knockout serum replacement medium on mitomycin C (Sigma-Aldrich) treated mouse embryonic fibroblasts (MEFs) feeder layers. The standard 20% knockout serum replacement medium contained 20% KO serum replacement (Invitrogen), 1% nonessential amino acids (at a dilution of 1:100 of 100× stock solution purchased from Invitrogen), 1 mM L-glutamine (Invitrogen), Dulbecco's modified Eagle's medium (DMEM/F12) (Invitrogen), 0.1 mM B-mercaptonethanol (Sigma-Aldrich), and 4 ng/ml FGF-2 (Sigma-Aldrich). The medium was changed every day and hESCs were passaged every 7 days.

Human embryonic stem cell colonies were treated with dispase (0.5 mg/ml, Invitrogen) to remove colonies from mouse embryonic feeder (MEF feeder) layers. The colonies were cultured in an ultra-low attachment dish (Costar) for 6 days in N2 medium [(Invitrogen) and described in detail in the appendix] consisting of DMEM/F12 [(Invitrogen) and described in detail in the appendix], nonessential amino acids (a 100× dilution, 1:100, purchased from Invitrogen) sodium pyruvate [(Invitrogen), 100× dilution], 1:100 N2 supplement [100× dilution purchased from Invitrogen] and described in detail in the appendix], and Fibroblast growth factor (FGF-2) (Sigma, 8 ng/ml) to form Embryoid Bodies (EBs).

At day 7, EBs were attached by using N2 medium supplement, described above with laminin (1 μg/ml, Invitrogen) on Poly-L-ornithine (PLO) (15 μg/ml, Sigma-Aldrich) coated surfaces.

After EBs formation, the stock solution of each of the ferric chloride, ferrous chloride, ferric citrate, citric acid, ammonium ferric citrate and ATP and were added to the EBs and changed every two days with the stock solution or alternatively, with media supplemented with the ferric chloride, ferrous chloride, ferric citrate, citric acid, ammonium ferric citrate and ATP.

Neural differentiation was monitored using beta-tubulin III, choline acetyltranserase (ChAT) and Olig2 marker expression using methods known in the art and described in Chao et al. (2009) supra.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### APPENDIX

D-MEM/F-12 (1×) liquid 1:1
Contains 15 mM HEPES buffer, L-glutamine and pyridoxine HCl.

### Catalog Number(s): 11330032, 11330057, 11330099, 11330107

| | | | |
|---|---|---|---|
| | | Concentration | |
| COMPONENTS | Molecular Weight | (mg/L) | mM |

Amino Acids

| | | | |
|---|---|---|---|
| Glycine | 75 | 18.75 | 0.25 |
| L-Alanine | 89 | 4.45 | 0 05 |
| L-Arginine hydrochloride | 211 | 147.5 | 0.699052 |
| L-Asparagine-H2O | 150 | 7.5 | 0.05 |
| L-Aspartic acid | 133 | 6.65 | 0.05 |
| L-Cysteine hydrochloride-H2O | 176 | 17.56 | 0.099773 |
| L-Cystine 2HCl | 313 | 31.29 | 0.099968 |
| L-Glutamic Acid | 147 | 7.35 | 0.05 |
| L-Glutamine | 146 | 365 | 2.5 |
| L-Histidine hydrochloride-H2O | 210 | 31.48 | 0.149905 |
| L-Isoleucine | 131 | 54.47 | 0.415802 |
| L-Leucine | 131 | 59.05 | 0.450763 |
| L-Lysine hydrochloride | 183 | 91.25 | 0.498634 |
| L-Methionine | 149 | 17.24 | 0.115705 |
| L-Phenylalanine | 165 | 35.48 | 0.21503 |
| L-Proline | 115 | 17.25 | 0.15 |
| L-Serine | 105 | 26.25 | 0.25 |
| L-Threonine | 119 | 53.45 | 0.44916 |
| L-Tryptophan | 204 | 9.02 | 0.044216 |
| L-Tyrosine disodium salt dihydrate | 261 | 55.79 | 0.213755 |
| L-Valine | 117 | 52.85 | 0.451709 |

Vitamins

| | | | |
|---|---|---|---|
| Biotin | 244 | 0.0035 | 0.000014 |
| Choline chloride | 140 | 8.98 | 0.064143 |
| D-Calcium pantothenate | 477 | 2.24 | 0.004696 |
| Folic Acid | 441 | 2.65 | 0.006009 |
| Niacinamide | 122 | 2.02 | 0.016557 |
| Pyridoxine hydrochloride | 206 | 2 | 0.009709 |
| Riboflavin | 376 | 0.219 | 0.000582 |
| Thiamine hydrochloride | 337 | 2.17 | 0.006439 |
| Vitamin B12 | 1355 | 0.68 | 0.000502 |
| i-Inositol | 180 | 12.6 | 0.07 |

Inorganic Salts

| | | | |
|---|---|---|---|
| Calcium Chloride (CaCl2) | 111 | 116.6 | 1.05045 |
| (anhyd.) | | | |
| Cupric sulfate (CuSO4—5H2O) | 250 | 0.0013 | 0.000005 |
| Ferric Nitrate (Fe(NO3)3″9H2O) | 404 | 0.05 | 0.000124 |
| Ferric sulfate (FeSO4—7H2O) | 278 | 0.417 | 0.0015 |
| Magnesium Chloride (anhydrous) | 95 | 28.64 | 0.301474 |
| Magnesium Sulfate (MgSO4) | 120 | 48.84 | 0.407 |
| (anhyd.) | | | |
| Potassium Chloride (KCl) | 75 | 311.8 | 4.157333 |
| Sodium Bicarbonate (NaHCO3) | 84 | 1200 | 14.285714 |
| Sodium Chloride (NaCl) | 58 | 6995.5 | 120.61207 |
| Sodium Phosphate dibasic | 142 | 71.02 | 0.500141 |
| (Na2HPO4) anhydrous | | | |
| Sodium Phosphate monobasic | 138 | 62.5 | 0.452899 |
| (NaH2PO4—H2O) | | | |
| Zinc sulfate (ZnSO4—7H2O) | 288 | 0.432 | 0.0015 |

Other Components

| | | | |
|---|---|---|---|
| D-Glucose (Dextrose) | 180 | 3151 | 17.505556 |
| HEPES | 238 | 3574.5 | 15.018908 |
| Hypoxanthine Na | 159 | 2.39 | 0.015031 |
| Linoleic Acid | 280 | 0.042 | 0.00015 |
| Lipoic Acid | 206 | 0.105 | 0.00051 |
| Phenol Red | 376.4 | 8.1 | 0.02152 |
| Putrescine 2HCl | 161 | 0.081 | 0.000503 |
| Sodium Pyruvate | 110 | 55 | 0.5 |
| Thymidine | 242 | 0.365 | 0.001508 |

### N-2 Supplement (100×) Liquid

### For the Growth and Expression of Post-Mitotic Neurons and Tumor Cells of Neuronal Phenotype.

N-2 supplement is a chemically defined, 100× concentrate of Bottenstein's N-2 formulation. This supplement is recommended for the growth and expression of neuroblastomas and for the survival and expression of post-mitotic neurons in primary cultures from both the peripheral nervous system (PNS) and the central nervous system (CNS). It can be used as a substitute for the N-1 Bottenstein formulation. N-2 medium appears to be selective for neuronal cell lines and does not support the growth of nonneuronal cell lines. (In Current Methods in Cellular Neurobiology: Volume 4, edited by Jeffery Barker, John Wiley & Sons, Inc., 1983.)

### Catalog Number(s): 17502014, 17502030, 17502048

| | | | |
|---|---|---|---|
| | Molecular | Concentration | |
| COMPONENTS | Weight | (mg/L) | mM |

Proteins

| | | | |
|---|---|---|---|
| Human Transferrin (Holo) | 10000 | 10000 | 1 |
| Insulin Recombinant Full Chain | 5807.7 | 500 | 0.086093 |

Other Components

| | | | |
|---|---|---|---|
| Progesterone | 314.47 | 0.63 | 0.002003 |
| Putrescine | 161 | 1611 | 10.006211 |
| Selenite | 173 | 0.52 | 0.003006 |

### MEM Non-Essential Amino Acids Solution 10 mM (100×) Liquid

Prepared in distilled water.

### pH: 1.5 to 1.7 Storage Condition: 2C to 8C

### Catalog Number(s): 11140-035, 11140035, 11140043, 11140050, 11140068, 11140076

| | | | |
|---|---|---|---|
| COMPONENTS | Molecular Weight | Concentration (mg/L) | mM |

Amino Acids

| | | | |
|---|---|---|---|
| Glycine | 75 | 750 | 10 |
| L-Alanine | | 890 | |
| L-Asparagine | | 1320 | |
| L-Aspartic acid | | 1330 | |
| L-Glutamic Acid | | 1470 | |
| L-Proline | | 1150 | |
| L-Serine | | 1050 | |

What is claimed is:

1. A neuronal growth media supplement composition comprising independently the same or different amount of from about 10 nM to about 100 μM of ferric chloride, ferrous chloride, ferri citrate, citric acid ammonium ferric citrate and ATP.

2. A composition for differentiating an isolated embryonic stem cell, an induced Pluripotent Stem Cell, a parthenogentic stem cell, or an isolated embryoid body into neuronal progenitor cells comprising independently the same or different amount of from about 10 nM to about 100 μM of each of ferric chloride, ferrous chloride, ferri citrate, citric acid ammonium ferric citrate and ATP and an effective amount of growth media.

3. The composition of claim 1 or 2, wherein the isolated embryonic stem cell is one or more of an induced Pluripotent stem cell or an embryoid body

4. The composition of claim 1 or 2, wherein the isolated stem cell is of animal origin.

5. The composition of claim 1 or 2, wherein the isolated stem cell is one or more of a human cell, a simian cell, or a murine cell.

6. The composition of claim 1 or 2, wherein the isolated embryonic stem cell is an isolated human embryonic stem cell.

7. A composition comprising the composition of claim 1 or 2, and a carrier.

8. The composition of claim 7, wherein the carrier is a pharmaceutically acceptable carrier.

9. An in vitro method for differentiating one or more of a stem cell from the group of an isolated embryonic stem cell, an induced Pluripotent stem cell, a parthenogenetic stem cell or an isolated embryoid body into a population of neural progenitor cells or neurons comprising contacting an effective amount of a composition of claim 1 with the stem cell, for an effective amount of time, thereby differentiating the stem cell.

10. An in vitro method for differentiating one or more of a stem cell from the group of an isolated embryonic stem cell, an induced Pluripotent stem cell, a parthenogenetic stem cell or an isolated embryoid body into a population of neural progenitor cells and/or neurons comprising contacting the cell from about 10 nM to about 100 μM of ferric chloride, ferrous chloride, ferri citrate, citric acid ammonium ferric citrate and ATP, wherein the amounts ferric chloride, ferrous chloride, ferri citrate, citric acid ammonium ferric citrate and ATP are independently the same or different.

11. The method of claim 9, wherein the population is substantially homogeneous.

12. The method of claim 10, wherein the population is substantially homogeneous.

13. A population of cells produced by the method of claim 11.

14. A population of cells produced by the method of claim 12.

15. The population of claim 13, further comprising an endogenous polynucleotide or polypeptide.

16. The population of claim 14, further comprising an endogenous polynucleotide or polypeptide.

17. The population of claim 13, further comprising a carrier or a biological scaffold.

18. The population of claim 14, further comprising a carrier or a biological scaffold.

19. A method for treating a neurodegenerative disorder comprising administering to a subject in need of such treatment an effective amount of the population of 13.

20. A method for treating a neurodegenerative disorder comprising administering to a subject in need of such treatment an effective amount of the population of claim 14.

21. A kit for use in preparing a population of neuronal progenitor cells comprising an effective amount of the composition of claim 1 or 2 and instructions for use of the composition.

22. A method for assaying a potential agent for the ability to affect cell migration, growth and/or differentiation of an isolated stem cell, comprising contacting at least one isolated stem cell with a composition of claim 1 or 2 and the agent, and culturing the cell under conditions that favor division of the at least one cell into a population of cells and observing the cell growth for the agent's effect on the cell's migration, growth and/or differentiation.
